(19) United States
(12) Patent Application Publication
(10) Pub. No.: US 2018/0221590 A1
(43) Pub. Date: Aug. 9, 2018

(54) APPARATUS FOR SAFELY DETACHING A HYPODERMIC NEEDLE FROM A SYRINGE

(71) Applicant: Rajpal SUBHASH, Toronto (CA)

(72) Inventor: Rajpal SUBHASH, Toronto (CA)

(21) Appl. No.: 15/427,084

(22) Filed: Feb. 8, 2017

(51) Int. Cl. A61M 5/32

(52) U.S. Cl. CPC A61M 5/3205; A61M 2209/04

(57) ABSTRACT

An apparatus for safely detaching a hypodermic needle from a syringe. The apparatus includes a body comprising a head portion, a stem portion; a shield coupled to the stem portion of the body, the shield comprising a base having an aperture shaped and dimensioned to receive a shaft of a hypodermic needle and grip a hub of the hypodermic needle. The apparatus also includes a plunger coupled to the body and moveable relative to the body along a longitudinal axis that passes through the plunger, the body and the shield, the plunger comprising a head and a stem, the stem configured to receive the shaft of the hypodermic needle when the hypodermic needle is inserted into the aperture of the base and the aperture of the base grips the hub of the hypodermic needle.

### FIELD

The present disclosure relates to an apparatus for safely detaching a hypodermic needle from a syringe and facilitates dispensing of the hypodermic needles into a disposal container.

### BACKGROUND

Luer lock and luer slip syringes are used to administer drugs to persons and/or animals. After such syringes are used to administer drugs, the used needle of the syringe is removed and dispensed into a disposal container. To remove the used needle from the syringe, a user, such as a healthcare worker, places a cap over the needle of the syringe, twists the cap to separate the needle from a tip of the syringe, and then places the separated needle into a container for disposal. Placing the cap over the used needle and separating the needle from the tip of the syringe can expose a user to the risk of being pricked by the needle (commonly referred to as a needle prick).

### BRIEF DESCRIPTION OF THE DRAWINGS

Embodiments of the present invention will be described, by way of example, with reference to the drawings and to the following description, in which:

FIG. 1 is an perspective view of an apparatus for detaching a hypodermic needle from a syringe in accordance with an embodiment;

FIG. 2 is an exploded perspective view of the apparatus of FIG. 1;

FIG. 3 is a perspective view of the plunger of the apparatus of FIG. 1;

FIG. 4 is a front view of the apparatus of FIG. 1;

FIG. 5 is a side view of the apparatus of FIG. 1;

FIG. 6 is a cross-sectional view of the apparatus of FIG. 1;

FIG. 7 is a bottom view of the apparatus of FIG. 1;

FIG. 8 is a perspective view of the apparatus of FIG. 1 in which a needle of a syringe is inserted into the apparatus;

FIG. 9 is an perspective view of the apparatus of FIG. 1 in which the needle is detached from the syringe;

FIG. 10 is an perspective view of the apparatus of FIG. 1 in which the needle is being ejected from the apparatus;

### DETAILED DESCRIPTION OF THE PREFERRED EMBODIMENTS

For simplicity and clarity of illustration, reference numerals may be repeated among the figures to indicate corresponding or analogous elements. Numerous details are set forth to provide an understanding of the embodiments described herein. The embodiments may be practiced without these details. In other instances, well-known methods, procedures, and components have not been described in detail to avoid obscuring the embodiments described. The description is not to be considered as limited to the scope of the embodiments described herein.

The present disclosure generally relates to an apparatus for detaching a hypodermic needle from a syringe, such as for example, a luer lock or luer slip syringe, and facilitates dispensing of the hypodermic needles into a disposal container, while mitigating the risk of a user suffering a needle prick.

According to one aspect of an embodiment, an apparatus for detaching a hypodermic needle from a syringe. The apparatus includes a body comprising a head portion, a stem portion; a shield coupled to the stem portion of the body, the shield comprising a base having an aperture shaped and dimensioned to receive a shaft of a hypodermic needle and grip a hub of the hypodermic needle. The apparatus also includes a plunger coupled to the body and moveable relative to the body along a longitudinal axis that passes through the plunger, the body and the shield, the plunger comprising a head and a stem, the stem configured to receive the shaft of the hypodermic needle when the hypodermic needle is inserted into the aperture of the base and the aperture of the base grips the hub of the hypodermic needle.

The plunger may be moveable relative to the body along the longitudinal axis between a needle receiving position in which an end of the stem of the plunger is adjacent to the aperture in the base of the shield and an ejection position in which the end of the stem of the plunger is spaced from the aperture in the base of the shield.

The plunger may be disposed within the body.

The body may further comprise a grasping portion disposed between the head portion and the stem portion to facilitate grasping of the body.

The grasping portion may be tapered from the head portion to the stem portion.

The plunger may be disposed in the body with at least a portion the head of the plunger disposed within the head portion of the body, and the stem is disposed within the stem portion of the body.

The plunger may be coupled to the body and moveable relative to the body along the longitudinal axis by a track.

The plunger may be coupled to the body and moveable relative to the body along the longitudinal axis by a pair of opposing tracks.

The shield may further comprise a stem housing extending substantially perpendicular away from the base, the stem housing configured to house the stem of the plunger therein.

Each of the pair of tracks may comprise a slot formed in the stem of the plunger and a rib formed on a stem housing of the body.

Each respective rib may be shaped and dimensioned to be received and slideable within the corresponding slot.

The stem portion of the body may be disposed over and surrounds the stem housing of the shield.

The stem portion of the body may be integrally coupled to the base of the shield.

The stem of the plunger may further comprise an inner tube configured to receive at least a portion of the shaft of the hypodermic needle when the hypodermic needle is inserted into the aperture of the base and gripped by the aperture of the base.

The stem of the plunger may further comprise an outer tube concentric with the inner tube.

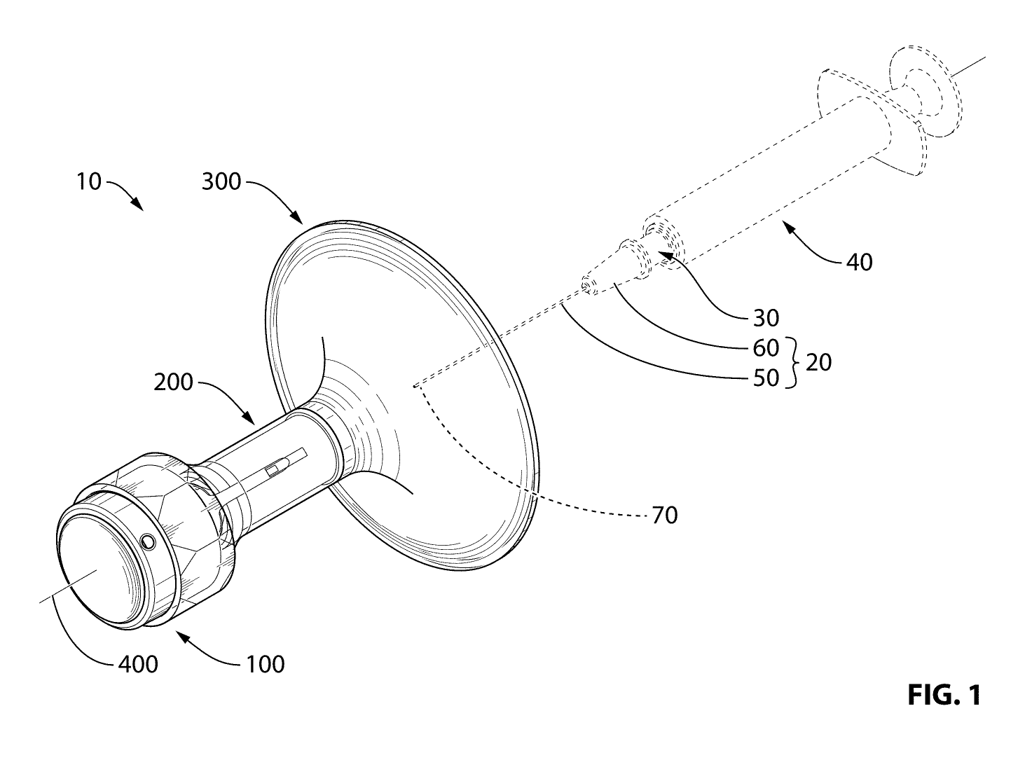

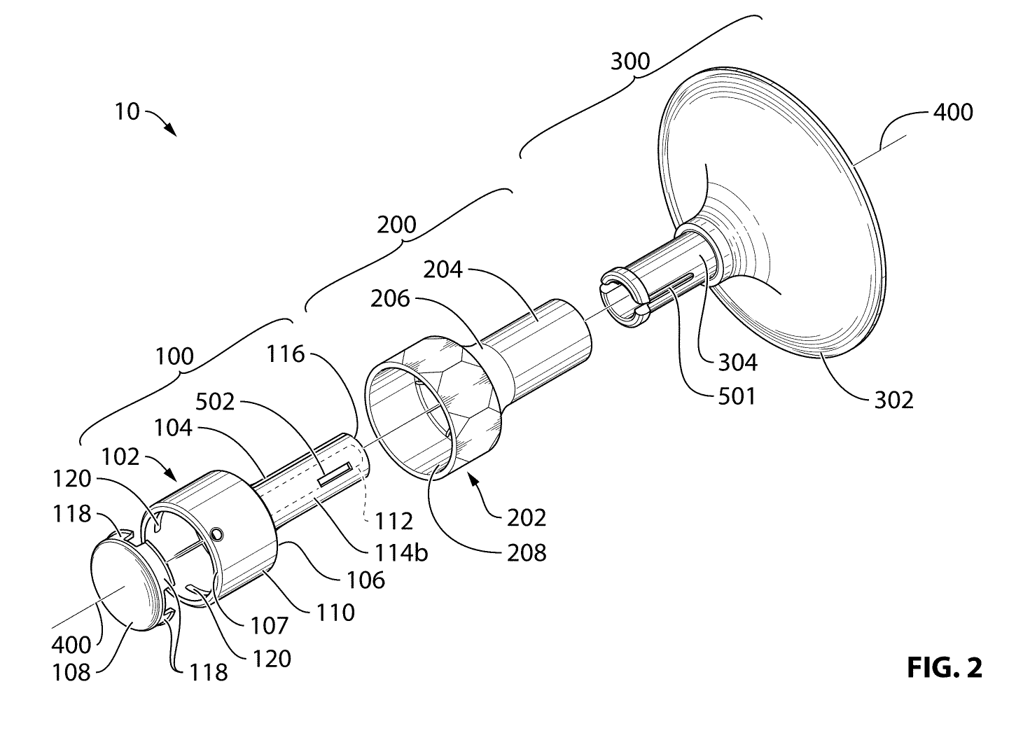

FIG. 1 to FIG. 7 show an example embodiment of an apparatus 10 for safely detaching a hypodermic needle 20 (hereinafter referred to as needle 20) from a tip 30 of syringe 40. The apparatus 10 includes a plunger 100, a body 200, a shield 300. A longitudinal axis 400 passes through the plunger 100, the body 200, and the shield 300. The plunger 100 is coupled to the body 200 and moveable relative to the body 200. The needle 20 includes a shaft 50 and a hub 60.

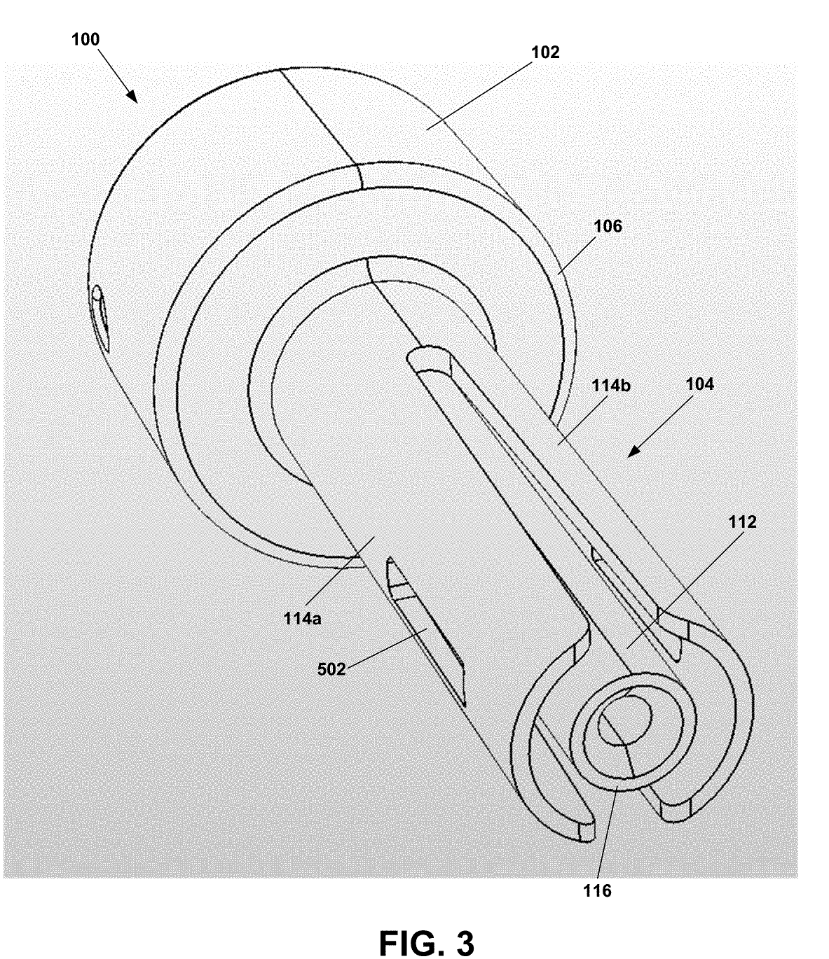

The plunger 100 includes a head 102 and a stem 104. The head 102 includes an interior end 106, an exterior end 107, a cap 108, and a sidewall 110 that extends between the interior end 106 and the exterior end 107. The stem 104 extends away from the interior end 106 of the head 102 along the longitudinal axis 400. The stem 104 comprises an inner tube 112 (illustrated in FIG. 3) and a pair of outer arms 114a, 114b (illustrated in FIG. 3). Each outer arm 114a, 114b extends away from the interior end 106 of the head 102 along opposing sides of the inner tube 112 such that inner tube 112 is disposed between the pair of outer arms 114a, 114b. In other words, the inner tube 112 and the pair of outer arms 114a, 114b are concentric about the longitudinal axis 400. The inner tube 112 of the stem 104 has an open end 116 for receiving the shaft 50 of the needle 20. The inner tube 112 is sized and dimensioned to receive and retain the shaft 50 of the needle 20 therein, as described in further detail below.

In the embodiment of the apparatus 10 shown in FIG. 1 to FIG. 7, the exterior end 107 of the plunger 100 is open and the cap 108 of the plunger 100 is removable from the head 102 of the plunger 100. The cap 108 includes tabs 118 and the exterior end 107 includes corresponding slots. The tabs 118 fit into and attach to corresponding slots 120 in the exterior end 107 of the head 102 to form an interference fit between the cap 108 and the exterior end 107 of the head 102. In alternative embodiments, the cap 108 is configured to be inserted into the exterior end 107 of the head 102 and snap fit to the exterior end 107 of the head 102. In still other embodiments, the cap 108 is integrally formed with the exterior end 107 of the head 102. In still other embodiments, the cap 108 may be omitted and the exterior end 107 of the head 102 is open.

It will be appreciated that in the embodiment shown in FIG. 1 to FIG. 7, the plunger 100 includes a head 102 and a stem 104 each having a cylindrical cross-section. However, in alternative embodiments, the plunger 100 has any suitable shape that fits within the body 200.

The body 200 includes a head portion 202, a stem portion 204, and a grasping portion 206 disposed between the head portion 202 and the stem portion 204. In the embodiment shown in FIG. 1 to FIG. 7, the body 200 is hollow and the head portion 202, the stem portion 204 and the grasping portion 206 each have a substantially cylindrical cross-section. The head portion 202 has an open end 208 having a first diameter for retaining the head 102 of the plunger 100 therein. The stem portion 204 has a second diameter that is smaller than the first diameter. The grasping portion 206 is tapered from the head portion 202 to the stem portion 204 to facilitate grasping between two adjacent fingers of a hand of a user (not shown). It will be appreciated that although the head portion 202, the stem portion 204 and the grasping portion 206 of the body 200 each have a substantially cylindrical cross-section in the embodiment shown in FIG. 1 to FIG. 7, in other alternative embodiments, the body 200 may each have any suitable shape.

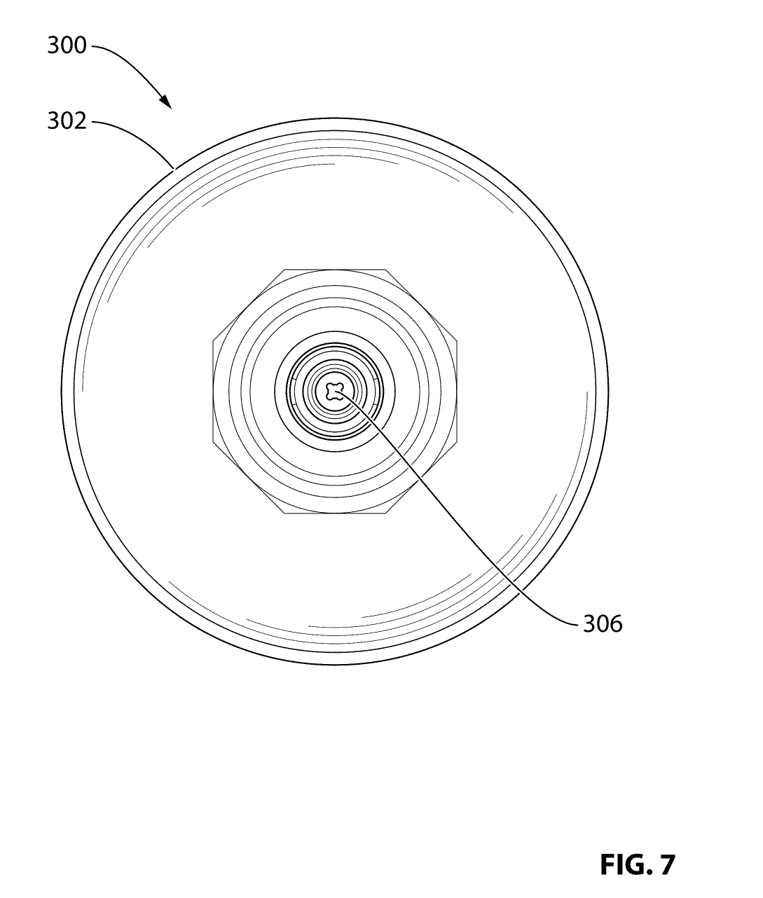

The shield 300 includes a base 302 and a stem housing 304. The base 302 includes an aperture 306 (FIG. 7) shaped and dimensioned to receive a bevel 70 of the shaft 50 of the needle 20 and to enable the bevel 70 and the shaft 50 of the needle 20 to pass through the aperture 306. The aperture 306 is also shaped and dimensioned to grip the hub 60 of the needle 20 as described in further detail below. The stem housing 304 extends away from the base 302 substantially perpendicular to the base 302. The stem housing 304 has a substantially cylindrical cross-section and is sized and shaped to fit inside the stem portion 204 of the body 200. In other words, the stem portion 204 of the body 200 surrounds the stem housing 304 of the shield 300. The stem housing 304 is also hollow, a portion of the stem 104 of the plunger 100 is disposed within the stem housing 304. In other words, a portion of inner tube 112 and the pair of outer arms 114a, 114 of the stem 104 of the plunger 100 reside inside the stem housing 304. It will be appreciated that in the embodiment depicted in FIG. 1 to FIG. 7, the base 302 of the shield 300 has an arcuate shape to enable the apparatus 10 to rest on a planar surface. However, in alternative embodiments, the base 302 may have any suitable shape. In alternative embodiments, the stem housing 304 has any suitable shape for receiving at least a portion of the stem 104 of the plunger 100.

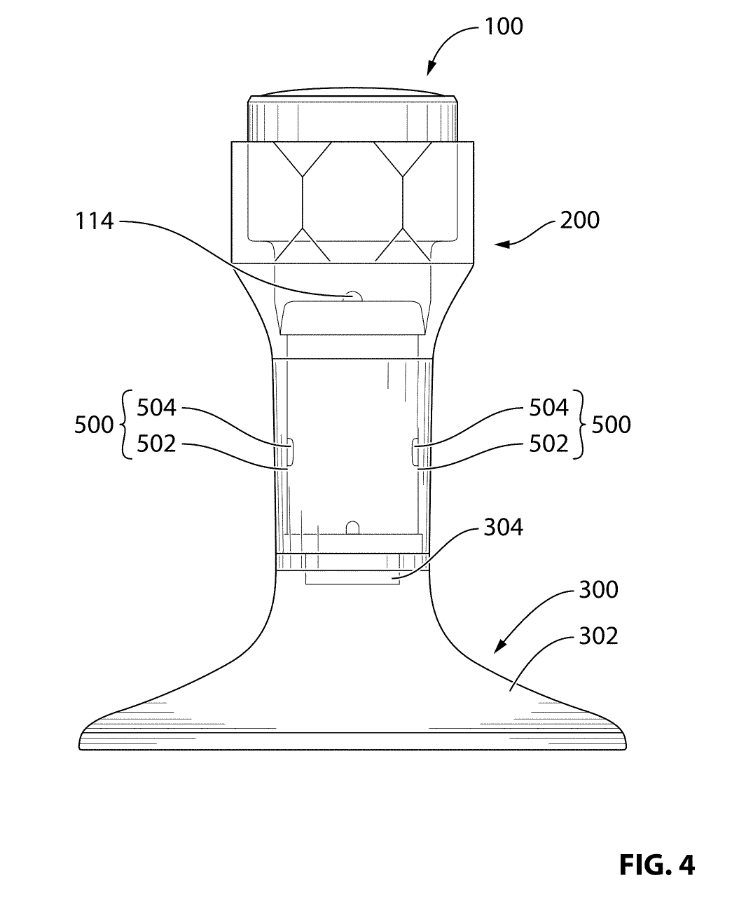

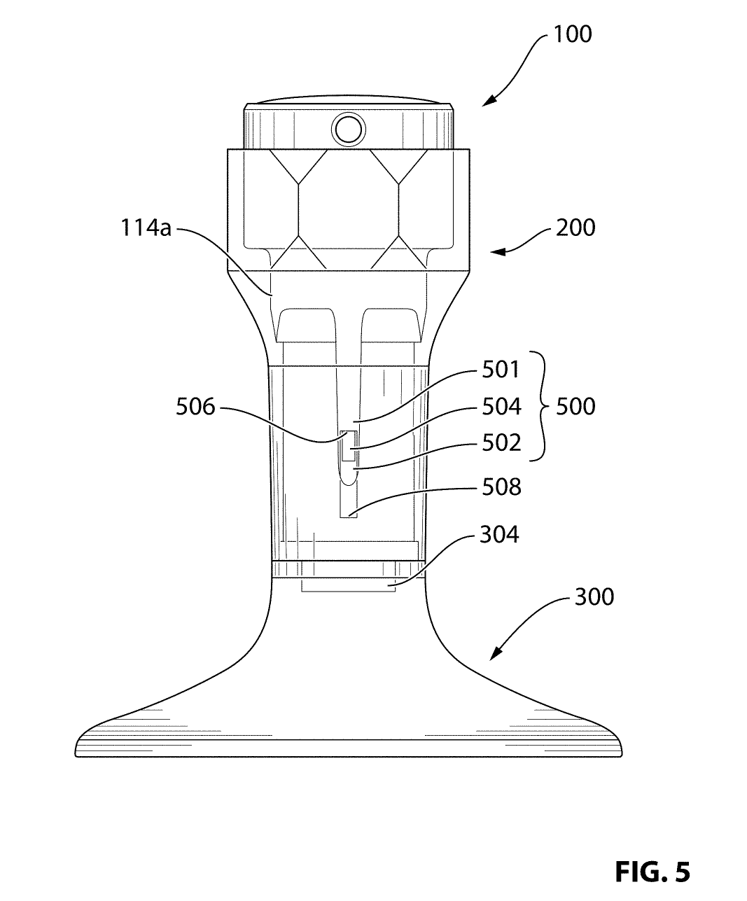

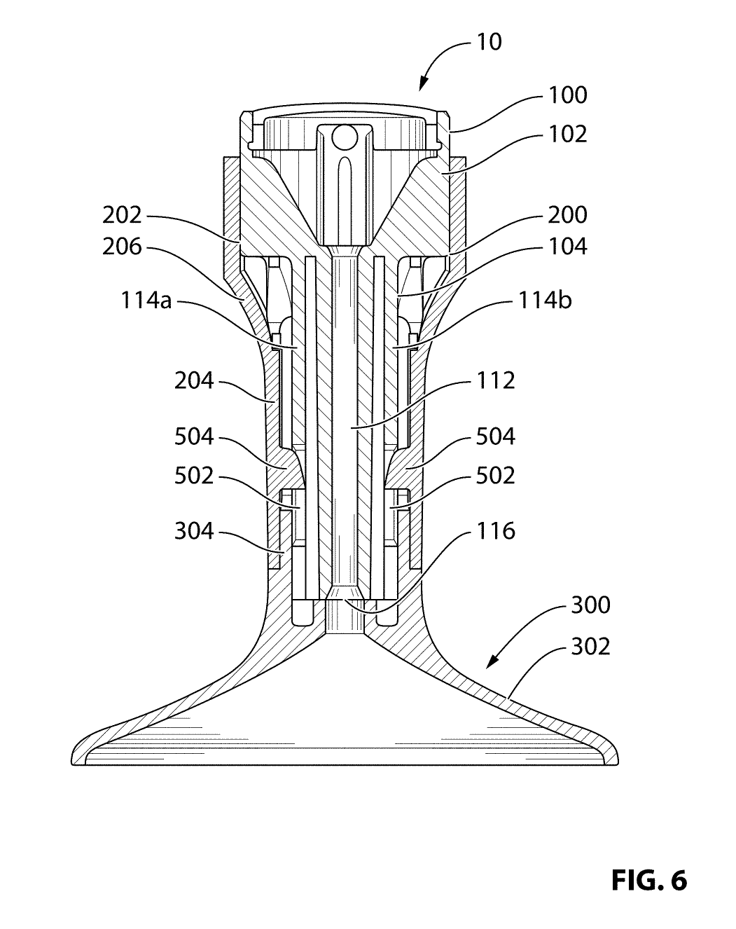

Referring to FIG. 4, FIG. 5, and FIG. 6, a front view, a side view, and a cross-sectional view of the apparatus 10 of FIG. 1 are shown. As shown in FIG. 4, FIG. 5, and FIG. 6, the plunger 100 is disposed within the body 200 such that the head 102 resides within the head portion 202 of the body 200 and the stem 104 resides within the stem portion 204 of the body 200. The head 102 rests on a ledge formed between the head portion 202 and the grasping portion 206 of the body 200. The plunger 100 is coupled to and moveable relative to the body 200 by a pair of tracks 500. Each track 500 comprises a channel 501 formed in the stem housing 304 of the shield 300, a slot 502 formed on the pair of outer arms 114a, 114b of the stem 104 of the plunger 100, and a rib 504 formed on an inner sidewall (not shown) of the stem portion 204 of the body 200. Each respective rib 504 extends substantially perpendicular away from the stem portion 204 of the body 200, through the respective channel 501 of the stem housing 304, and then into the respective slot 502 in the stem 104. Each respective rib 504 is shaped and dimensioned to be received within its respective slot 502 and its respective channel 501. The plunger 100 is moveable within the body 200 such that each respective rib 504 slides within its corresponding slot 502 and its corresponding channel 501. The plunger 100 is moveable relative to the body 200 between a first position (e.g. a needle receiving position) for receiving the shaft 50 of the needle 20 of the syringe 40 and a second position (e.g. an ejection position) for ejecting the needle 20 of the syringe 40 (e.g. the shaft 50 and the hub 60 of the needle 20). In the first position (e.g. the needle receiving position), the open end 116 of the inner tube 112 of the stem 104 of the plunger 100 is adjacent the aperture 306 of the base 302 of the shield 300 and each respective rib 504 is proximate a first end 506 (FIG. 5) of each respective slot 502. In the second position (e.g. the ejection position), the open end 116 of the inner tube 112 of the stem 104 of the plunger 100 is spaced from the aperture 306 of the base 302 of the shield and each respective rib 504 is proximate a second end 508 (FIG. 5) of each respective slot 502. The track 500 limits the travel of the plunger 100 relative to the body 200.

It will be appreciated that some alternative embodiments, in the first position, each respective rib 504 is proximate or abuts the first end 506 of each respective slot 502, and in the second position, each respective rib 504 is positioned between the first end 506 and the second end 508 of each respective slot 502. In some embodiments, the distance of travel of the plunger 100 relative to the body 200 is dependent on a length and taper profile of the hub 60 of the syringe 40.

Although the plunger 100 is coupled to the body 200 and moveable relative to the body 200 by a track 500 in the embodiment shown in FIG. 1 to FIG. 7, in other alternative embodiments, the plunger 100 is coupled to body 200 and moveable relative to the body 200 by any suitable mechanical mechanism, such as, for example, a screw and nut mechanism in which the plunger 100 rotates within the body 200 towards the aperture 306 when the plunger 100 is turned in one direction (e.g. clockwise or counter clockwise) and away from the aperture 306 when the plunger 100 is turned in an opposite direction (e.g. counter clockwise or clockwise). In still other embodiments, the track 500 may be omitted and the plunger 100 and the body 200 are coupled together by a sliding fit such that the plunger 100 fits inside the body 200 and slides towards and away from the aperture 306.

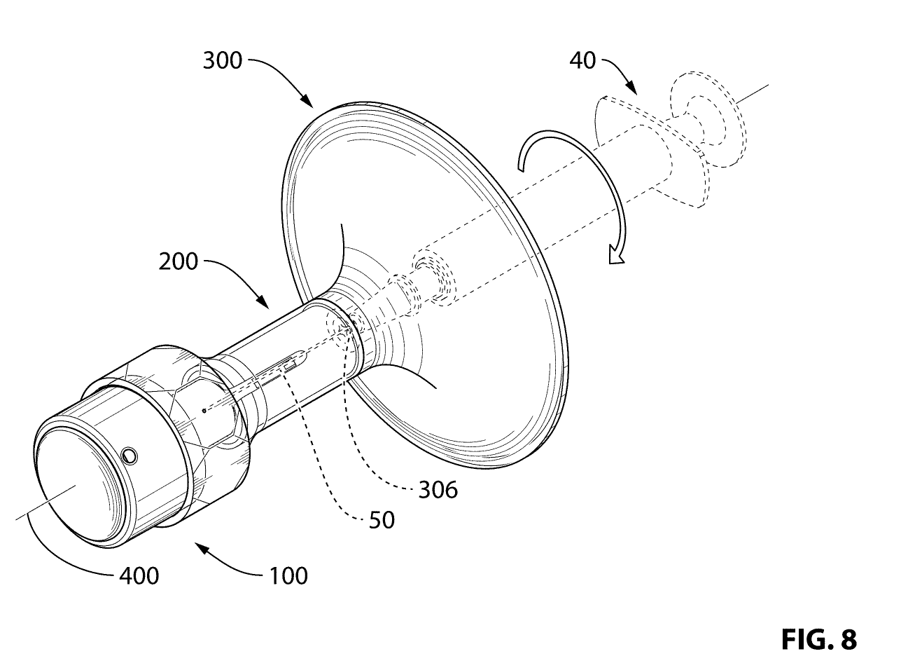

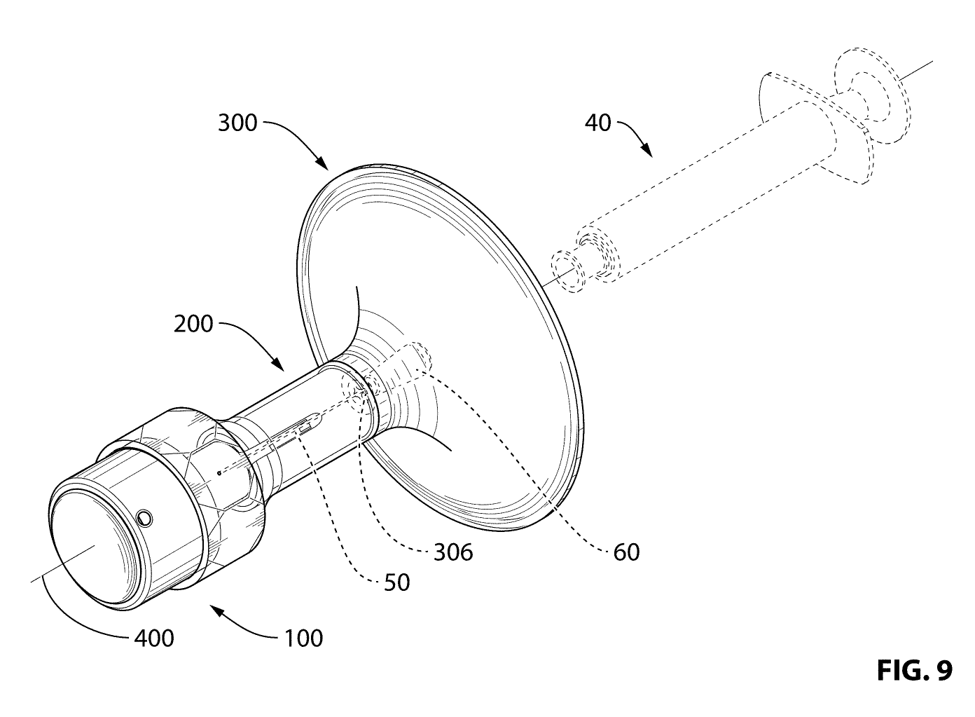

The operation of the apparatus 10 will now be described with reference to FIG. 8 to FIG. 10. To detach the needle 20 from a syringe 40, the shaft 50 of the needle 20 of the syringe 40 is inserted into the aperture 306 of the base 302 of the shield 300 such that the shaft 50 of the needle 20 enters the inner tube 112 of the stem 104. As the hub 60 approaches the aperture 306 of the shield and is inserted into thereto, the hub 60 of the needle 20 contacts the inner tube 112 of the stem 104 of the plunger 100 and the plunger 100 moves away from the base 302 of the shield 300 from the first position (e.g. the needle receiving position) in which the open end 116 of the inner tube 112 is adjacent the aperture 306 of the shield 300 and each respective rib 504 is located at the first end 506 of its corresponding slot 502 until the plunger 100 reaches the second position (e.g. the ejection position) in which the open end 116 of the inner tube 112 is spaced from the aperture 306 of the shield 300 and each respective rib 504 is located at the second end 508 of its corresponding slot 502, as illustrated in FIG. 8. When the aperture 306 grips the hub 60 of the needle 20 and the shaft 50 of the needle 20 is disposed inside the inner tube 112, the plunger 100 is at the second position, the needle 20 can be detached from the syringe 40. If the syringe is a luer-lock syringe, the needle 20 can be detached from the syringe by twisting on the syringe 40 either clockwise or counter clockwise direction, as illustrated in FIG. 9. Alternatively, if the syringe 40 is a luer-slip syringe, the needle 20 can be detached by pulling the syringe 40 and the apparatus 10 away from each other to release the hub 60 from the syringe 40. Once the hub 60 is released from the syringe 40, the hub 60 of the syringe 40 is remains gripped by the aperture 306 and the shaft 50 of the needle 20 is disposed inside the inner tube 112.

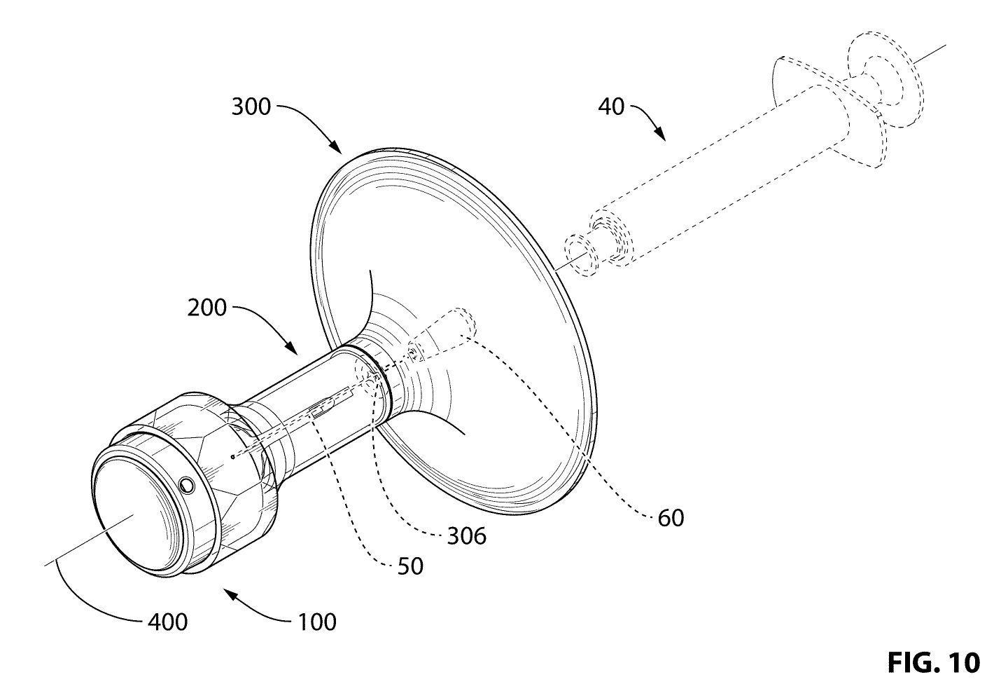

Once the needle 20 is detached from the syringe 40, the detached needle 20 (e.g. the shaft 50 and the hub 60 of the needle 20) can be ejected from the apparatus 10 and dispensed into a disposal container, as illustrated in FIG. 10. To eject the detached needle 20 from the apparatus 10, the plunger 100 is moved relative the body 200, by for example, a user pushing on the cap 108 of the plunger 100, towards the aperture 306 in the base 302 of the shield 300 from the second position to the first position. As the plunger 100 moves towards the aperture 306 in the base 302 of the shield 300, the inner tube 112 pushes on the hub 60 of the syringe 40 to release the grip of the aperture 306 on the hub 60, which causes the hub 60 to be released from the aperture 306 and the shaft 50 located in the inner tube 112 and the hub 60 of the detached needle 20 to be ejected from the apparatus 10.

Referring again to FIG. 1 to FIG. 7, the apparatus 10 is made of transparent or opaque plastics, such as, for example, polypropylene. However, in other embodiments, the apparatus 10 is made of any suitable thermoplastic, such as, for example, a thermoplastic elastomer. In still other embodiments, the apparatus 10 is manufactured by plastic moulding processes, such as injection moulding.

It will be appreciated that although the stem housing 304 of the shield 300 is disposed inside the stem portion 204 of the body 200 in the embodiment of the apparatus 10 depicted in FIG. 1 to FIG. 7, in alternative embodiments, the shield 300 does not include the stem housing 304 and the stem portion 204 of the body 200 is integrally coupled to the base 302.

In other alternative embodiments, the plunger 100 is coupled to an outer surface of the body 200 and moveable relative to the body 200.

In still other alternative embodiments the plunger 100 has a uniform diameter. In other words, the head 102 and the stem 104 have the same diameter. In this embodiment, the body 200 has a uniform diameter and is configured to receive the plunger 100 having the uniform therein. Thus, the body 200 does not include a grasping portion 206 in this embodiment.

In still other alternative embodiments, the stem 104 of the plunger 100 includes an outer tube (not shown) that surrounds the inner tube 112 rather than the pair of outer arms 114a, 114b. In this alternative embodiment, the inner tube 112 and the outer tube (not shown) are concentric about the longitudinal axis 400 and the outer tube (not shown) includes the slots 502.

The apparatus of the present invention facilitates the detachment of a needle from a syringe after the syringe has been used to dispense drugs while mitigating the risks of a user suffering a needle prick.

The described embodiments are to be considered in all respects only as illustrative and not restrictive. The scope of the claims should not be limited by the preferred embodiments set forth in the examples, but should be given the broadest interpretation consistent with the description as a whole. All changes that come with meaning and range of equivalency of the claims are to be embraced within their scope. The terms interior, exterior, inner, outer, are utilized herein to provide reference to the orientation of the apparatus 10 as shown in FIG. 4 and FIG. 5.

What is claimed is:

1. An apparatus for detaching hypodermic needles from syringes, the apparatus comprising:
a body comprising a head portion, a stem portion;
a shield coupled to the stem portion of the body, the shield comprising a base having an aperture shaped and dimensioned to receive a shaft of a hypodermic needle and grip a hub of the hypodermic needle; and,
a plunger coupled to the body and moveable relative to the body along a longitudinal axis that passes through the plunger, the body and the shield, the plunger comprising a head and a stem, the stem configured to receive the shaft of the hypodermic needle when the hypodermic needle is inserted into the aperture of the base and the aperture of the base grips the hub of the hypodermic needle.

2. The apparatus of claim 1, wherein the plunger is moveable relative to the body along the longitudinal axis between a needle receiving position in which an end of the stem of the plunger is adjacent to the aperture in the base of the shield and an ejection position in which the end of the stem of the plunger is spaced from the aperture in the base of the shield.

3. The apparatus of claim 1, wherein the plunger is disposed within the body.

4. The apparatus of claim 1, wherein the body further comprises a grasping portion disposed between the head portion and the stem portion to facilitate grasping of the body.

5. The apparatus of claim 4, wherein the grasping portion is tapered from the head portion to the stem portion.

6. The apparatus of claim 4, wherein the plunger is disposed in the body with at least a portion the head of the plunger disposed within the head portion of the body, and the stem is disposed within the stem portion of the body.

7. The apparatus of claim 1, wherein the plunger is coupled to the body and moveable relative to the body along the longitudinal axis by a track.

8. The apparatus of claim 1, wherein the plunger is coupled to the body and moveable relative to the body along the longitudinal axis by a pair of opposing tracks.

9. The apparatus of claim 8, wherein each track of the pair of tracks comprises a slot formed in the stem of the plunger and a rib formed on a stem portion of the body.

10. The apparatus of claim 9, wherein each respective rib is shaped and dimensioned to be received and slideable within the corresponding slot formed in the stem of the plunger.

11. The apparatus of claim 1, wherein the shield further comprises a stem housing extending substantially perpendicular away from the base, the stem housing configured to house the stem of the plunger therein.

12. The apparatus of claim 1, wherein the stem portion of the body is disposed over and surrounds the stem housing of the shield.

13. The apparatus of claim 1, wherein the stem portion of the body is integrally coupled to the base of the shield.

15. The apparatus of claim 1, wherein the stem of the plunger further comprises an inner tube configured to receive at least a portion of the shaft of the hypodermic needle when the hypodermic needle is inserted into the aperture of the base and gripped by the aperture of the base.

16. The apparatus of claim 15, wherein the stem of the plunger further comprises a pair of outer arms concentric with the inner tube.
